(19) United States
(12) Patent Application Publication
(10) Pub. No.: US 2010/0174363 A1
(43) Pub. Date: Jul. 8, 2010

(54) One Piece Prosthetic Valve Support Structure and Related Assemblies

(75) Inventor: Salvatore Castro, Milford, MA (US)

Correspondence Address: MCCARTER & ENGLISH, LLP STAMFORD, CANTERBURY GREEN, 201 BROAD STREET, 9TH FLOOR, STAMFORD, CT, 06901, US

(73) Assignee: EndoValve, Inc., Princeton, NJ (US)

(21) Appl. No.: 12/649,692

(22) Filed: Dec. 30, 2009

Related U.S. Application Data
(60) Provisional application No. 61/142,975, filed on Jan. 7, 2009.

(51) Int. Cl. A61F 2/24; B23P 13/04

(52) U.S. Cl. 623/2.11; 623/2.36; 29/557

(57) ABSTRACT

Valve prostheses are disclosed that are adapted for secure and aligned placement relative to a heart annulus. The valve prostheses may be placed in a non-invasive manner, e.g., via trans-catheter techniques. The valve prosthesis may include a support ring, a plurality of leaflet membranes mounted with respect to the support ring, and a plurality of retention clip elements. The support structure is advantageously formed from a tubular member or flat stock of shape memory metal. A delivery system for delivery of a valve prosthesis embodying the disclosed support structure is also disclosed.

### CROSS-REFERENCE TO RELATED APPLICATION

The present application claims priority benefit to a co-pending provisional application entitled “One Piece Prosthetic Valve Support Structure and Related Assemblies” which was filed on Jan. 7, 2009 and assigned Ser. No. 61/142,975. The entire content of the foregoing provisional patent application is incorporated herein by reference.

### BACKGROUND

1. Technical Field

The present disclosure is directed to advantageous valve prosthesis systems and associated methods/systems for placement of a heart valve prosthesis and, more particularly, to a one-piece mitral valve prosthesis that is adapted for secure and aligned placement relative to a heart annulus and associated methods/systems for placement thereof

2. Background Art

Heart valve regurgitation occurs when the heart valve does not close completely as a result of disease or injury. Mitral regurgitation due to ischemic and degenerative (prolapse) disease has been shown to contribute to left ventricular dilation and dysfunction due to remodeling, and is associated with increased rates of cardiac events and death. Currently, malfunctioning heart valves may be replaced with biologic or mechanical prostheses through open-heart surgery with the attendant significant risk of death, stroke, infection, bleeding, and complications due to the use of general anesthesia and cardiopulmonary bypass.

Based on the success of percutaneous balloon valvuplasty for mitral stenosis, investigators have explored other alternative methods to treat valvular heart disease without surgery. For example, Cribier et al. describe a balloon-expandable stent to which a biologic valve prosthesis is sewn. (See, “Percutaneous Transcatheter Implantation of an Aortic Valve Prosthesis for Calcific Aortic Stenosis,” Circulation, Dec. 10, 2002, pages 3006-3008.) The Cribier device is utilized to treat calcific aortic stenosis. Bonhoeffer et al. describe a similar stent approach with a bovine venous (jugular) valve inserted to treat pulmonic valve disease. (See, “Percutaneous Insertion of the Pulmonary Valve,” Journal of the American College of Cardiology, Vol. 39, No. 10, May 15, 2002, pages 1664-1669.) Others are developing repair techniques for mitral valve disease that involve placing a clip on the mitral leaflets (U.S. Pat. No. 6,629,534), cinching the mitral annulus from the coronary sinus (U.S. Pat. No. 6,537,314), or deploying an inflatable heart valve that is mechanically held in place (U.S. Pat. No. 5,554,185).

Norred (U.S. Pat. No. 6,482,228) discloses a percutaneous aortic valve replacement in which a heart valve prosthesis having ribs and a circular elastomeric canopy is folded for insertion into a catheter for delivery to the implantation region without surgery. Once in the ascending aorta, the body and leaflets of the heart valve prosthesis are opened like an umbrella by pulling on a central column of suture-like members. Hinge joints are used to create a miniature umbrella. However, the aortic valve prosthesis is anchored using a stent system that is extended in the ascending aorta to anchor the valve in the aortic channel above the biologic aortic valve. The suture-like members used to open the umbrella structure are deployed as part of the stent system. Such a design is not amenable to placement of the heart valve prosthesis at the location of the biologic valve.

Other stented heart valve prostheses are described in the art in which the anchoring system is a passive one that requires either balloon expandable stents or a self-expanding stent design. For example, such stented designs are described in U.S. Pat. No. 6,454,799, US 2002/0138138, U.S. Pat. No. 6,582,462, U.S. Pat. No. 6,458,153, U.S. Pat. No. 6,425,916, and U.S. Pat. No. 5,855,601. It will be appreciated that once these stented heart valve prostheses are deployed, they cannot be repositioned, refolded, or easily removed. Furthermore, the rigidity of the stent as it is deployed in calcified positions may allow for regurgitation around the outside of the stent, as has been seen in the early aortic valve deployments which utilize this design. It is also difficult to position these designs as one has to inflate a balloon in a moving column of blood while the heart is beating and one only gets one chance to accurately deploy it.

An additional difficulty occurs when deploying a stented heart valve in an annulus that is not thickened by calcium. The stent design lends itself slightly better to the aortic position where the height of the annulus has been increased and the width thickened by the presence of calcium in calcific aortic stenosis. However, when calcium is not present, as in other causes of aortic valve disease and in the mitral position, the stent may be difficult to anchor on the relatively thin annulus. Furthermore, the nature by which the stent folds on a balloon and then expands with plastic deformability limits the ratio of its initial to final size such that it will, by necessity, have a fairly large profile making percutaneous insertion via catheter more difficult in a valve annulus with a large diameter that has not been reduced by calcium deposition.

Herrmann et al. (US 2007/0016286) disclose a percutaneously inserted bistable heart valve prosthesis that may be folded inside a catheter for delivery to the patient's heart for implantation. The heart valve has an elastic annular ring, a body member having a plurality of legs, each leg connecting at one end to the annular ring, claws that are adjustable from a first position to a second position by application of external force so as to allow ingress of surrounding heart tissue into the claws in the second position, and leaflet membranes connected to the annular ring, the body member and/or the legs. The disclosed leaflet membranes have a first position for blocking blood flow therethrough and a second position for allowing blood flow therethrough. The heart valve is designed such that upon removal of the external force, the claws elastically revert to the first position so as to grip the heart tissue positioned within the claws, thereby holding the heart valve in place. The body member and claws may be integrated into a one-piece design. The heart valve so designed may be used as a prosthesis for the mitral valve, aortic valve, pulmonary valve, or tricuspid valve by adapting the annular ring to fit in a respective mitral, aortic, pulmonary, or tricuspid valve opening of the heart.

Machold et al. (US 2004/0127982) disclose an implant that is sized and configured to attach to the annulus of a dysfunctional heart valve. In use, the implant extends across the major axis of the annulus above and/or along the valve annulus. The implant reshapes the major axis dimension and/or other surrounding anatomic structures and is intended to restore a more functional anatomic shape and tension. Machold et al. contemplate a pair of struts that are joined by a rail and that carry other structures to enhance the anchorage and stabilization of the implant in the heart valve annulus. The anchoring mechanisms may be located below the plane of the annulus to engage infra-annular heart tissue adjoining the annulus in the ventricle and/or may be located at or above the plane of the annulus, to engage tissue on the annulus or in the atrium. Machold et al. further disclose that the struts may be used to simply locate the implant in the valve, imparting little or no force on their own. In this arrangement, the annulus reshaping forces of the Machold design emanate from the rail(s) above the commissures.

Under image guidance, the Machold et al. strut on the leading end of the implant is freed from a sheath and seated retrograde in the posterior commissure of the valve annulus. Anchoring structures or mechanisms associated with the strut are also placed into contact with adjoining tissue below and/or above the plane of the annulus. As shown in FIG. 25B, the delivery catheter maintains force on the leading strut within the posterior commissure as the sheath is withdrawn in line with the coaptation line in a posterior-to-anterior direction along the coaptation line. Similar structures for positioning an implant relative to an annulus are disclosed by Vazquez et al. (U.S. Pat. No. 6,287,339).

Despite efforts to date, a need remains for an improved heart valve prosthesis design that allows a low profile for insertion via a catheter but, in the absence of a balloon or stent, transforms to a large profile once deployed. A heart valve prosthesis design is also desired that can be deployed, folded, removed, and then redeployed so as to increase the safety as well as the preciseness of prosthesis deployment. Still further, a need remains for heart valve prosthesis design(s) that are easily and reliably fabricated, and that may be effectively aligned and/or oriented relative to the heart. Reliable and effective deployment systems and methods for such advantageous heart valve prostheses are also needed.

These and other needs are addressed by the disclosed prosthesis designs and deployment systems/methodologies, as will be apparent from the detailed description which follows.

### SUMMARY

Advantageous prosthetic valve support structures, valve systems and methods/systems for placement of valve prostheses are disclosed herein. In exemplary embodiments of the present disclosure, a mitral valve prosthesis is provided that is adapted for secure and aligned placement relative to a heart annulus. The disclosed valve prosthesis systems may be placed in a non-invasive manner, e.g., via trans-catheter techniques.

According to the present disclosure, an advantageous one-piece support structure is provided for use in supporting, rigidifying and retaining a replacement valve structure in situ. An exemplary support structure according to the present disclosure takes the form of a tripod and is adapted to support a plurality (typically three) valve leaflets. The one-piece support structure is advantageously fabricated from a single tubular member, e.g., a tubular member fabricated from a shape memory metal (e.g., Nitinol). Thus, in exemplary embodiments, a Nitinol tubular member is strategically cut, primarily along its longitudinal axis, to define requisite features for supporting, rigidifying and retaining a replacement valve structure, e.g., a replacement mitral valve structure.

More particularly, the disclosed tubular member is advantageously cut such that, when radially expanded, a support structure is defined that includes (i) a proximal mounting collar, (ii) three (3) support struts, (iii) a support ring, and (iv) three (3) retention clip elements. The mounting collar is adapted to interact with and be detachably mounted with respect to a delivery system. The support struts extend from the mounting collar and typically define a substantially arcuate or curved geometry. A slot region is defined in each support strut due to the lower portion of the retention clip element associated therewith. More particularly, through the cutting operation disclosed herein, retention clip elements are defined from the tubular member, and the lower portions of the retention clip elements are essentially cut from the support struts, thereby defining slot regions in the support struts.

The support ring extends circumferentially around the support structure and provide a rigidifying force to the support structure for effective positioning/functioning as a valve prosthesis. The support ring may be viewed as three distinct segments, each segment extending from support strut to support strut. Each segment is defined by downward deflection of side-by-side portions of the tubular member. An upstanding tab may be defined at (or near) the midpoint of each segment. The upstanding tabs may facilitate mounting of leaflet members with respect to support structure. The support ring offers a degree of flexibility/resilience during deployment, but when fully deployed, assumes a substantially rigid/fixed geometry relative to the annulus within which it is placed.

Retention clip elements are defined through longitudinal cuts applied to the tubular member. The retention clip elements typically define upper and lower portions which, together, define a substantially C-shaped clip geometry. The retention clip elements are generally designed to position/align the support structure relative to an annulus, e.g., a heart annulus, and to provide retention functionality with respect thereto. As noted above, the lower portion of each retention clip element is cut from a corresponding support strut (thereby defining a slot region in the support strut) and the upper portion of the retention clip element extends above the substantially horizontal plane defined by the support ring.

Leaflet members are secured to the support ring so as to define a valve prosthesis. The leaflet membranes may be fabricated from xenograft tissue, e.g., the valve leaflets may be fabricated from standard biologic or artificial prosthetic material, such as cryo- or chemically-preserved bovine pericardium or porcine heart valve tissue. Synthetic membrane materials may also be employed in the fabrication of the leaflet membranes, e.g., fiber-reinforced matrix materials. The leaflet membranes may be secured with respect to the support ring through conventional means, e.g., creation of an annulus and/or cuff that surrounds, in whole or in part, the support ring such that each of the plurality of leaflet membranes extends downwardly with respect to the support ring.

In use, the disclosed support structure permits a clinician to deliver, position and release a valve prosthesis in situ. The support structure is generally defined by applying the requisite cuts to a tubular member formed of a shape memory metal, e.g., Nitinol. Thereafter, the tubular member is deflected/expanded so as to assume a desired expanded configuration, i.e., to define the disclosed “support structure” a mounting collar, support struts, support ring and retention clip elements are in their desired relative orientation, and the shape memory metal/Nitinol is processed so as to “retain” such expanded configuration in memory. Thereafter, the support structure may be collapsed into a substantially tubular orientation, e.g., for trans-catheter delivery, and retained in such collapsed configuration, e.g., by placement within a delivery tube. When released from the delivery tube, the support structure automatically returns to its expanded configuration, thereby facilitating placement/positioning relative to a desired anatomical structure, e.g., a heart annulus.

The present disclosure also provides an advantageous delivery system for effective and reliable delivery of a valve prosthesis. In particular, the disclosed delivery system is effective for delivery of the disclosed support structure and associated leaflet membranes, i.e., a valve prosthesis that includes the disclosed support structure. Exemplary delivery systems according to the present disclosure include a body housing, a sheath advance housing and associated structures for effecting deployment of the valve prostheses, e.g., a valve prosthesis that includes the presently disclosed support structure.

Additional advantageous features, structures and functions associated with the disclosed valve prosthesis will be apparent from the description of exemplary embodiments which follows, particularly when read in conjunction with the accompanying figures.

### BRIEF DESCRIPTION OF THE FIGURES

To assist those of ordinary skill in the art in making and using the disclosed valve prosthesis system and associated deployment systems/methods, reference is made to the accompanying figures wherein:

FIG. 1 is perspective view of a tubular member to which has been applied a series of cuts to define an exemplary support structure according to the present disclosure;

FIG. 2 is a “laid-flat” side view of the tubular member of FIG. 1 further illustrating exemplary cuts applied thereto;

FIGS. 3A-3D provide a series of views of an exemplary support structure formed from the tubular member of FIGS. 1 and 2;

FIG. 4 is a perspective view of an exemplary delivery system for a support structure according to the present disclosure;

FIG. 5 is -16 provide a series of views of an exemplary delivery system according to the present disclosure.

### DESCRIPTION OF EXEMPLARY EMBODIMENT(S)

Advantageous valve support structures, valve prosthesis systems and deployment systems/methods are provided according to the present disclosure. The disclosed systems and methods permit surgeons/clinicians to improve heart valve function without invasive surgical intervention. Indeed, the disclosed valve prosthesis systems permit a heart valve prosthesis to be percutaneously delivered to a desired anatomical location. Once located in the desired anatomical region/locale, the disclosed valve prosthesis system facilitates secure and aligned placement of a heart valve prosthesis relative to a heart annulus. Percutaneous delivery of the disclosed heart valve prosthesis as disclosed herein provides for efficient and effective clinical placement of a heart valve prosthesis. The disclosed heart valve prosthesis and associated delivery techniques offer numerous clinical benefits, including enhanced valve function without the need to remove existing valve leaflets, an ability to effectively and efficiently deliver a valve prosthesis percutaneously, and an ability to position a valve prosthesis relative to an annulus to ensure proper orientation relative to anatomical features.

In addition, the design and manufacturing technique for the disclosed support structure offers significant advantages. For example, fabrication of the disclosed support structure from a single tubular component is effective, reliable and cost-effective, eliminating potential assembly and/or tolerance-related issues associated with combination of multiple components/parts. The disclosed support structure is generally fabricated from a tubular member that is formed from a shape memory metal, e.g., Nitinol. Programmed cutting tools may be employed to precisely deliver requisite cuts to the tubular member such that, upon expansion, a support structure having advantageous structural features and functionalities is effectively manufactured. Moreover, by processing the shape memory/Nitinol structure in its expanded configuration, the support structure may be provided with “memory” as to its expanding configuration. Once such “memory” is imparted to the disclosed support structure, further handling of the support structure in connection with anatomical delivery of a valve prosthesis may be achieved in reliance on the support structure re-assuming its expanded configuration, e.g., upon release from a delivery sheath.

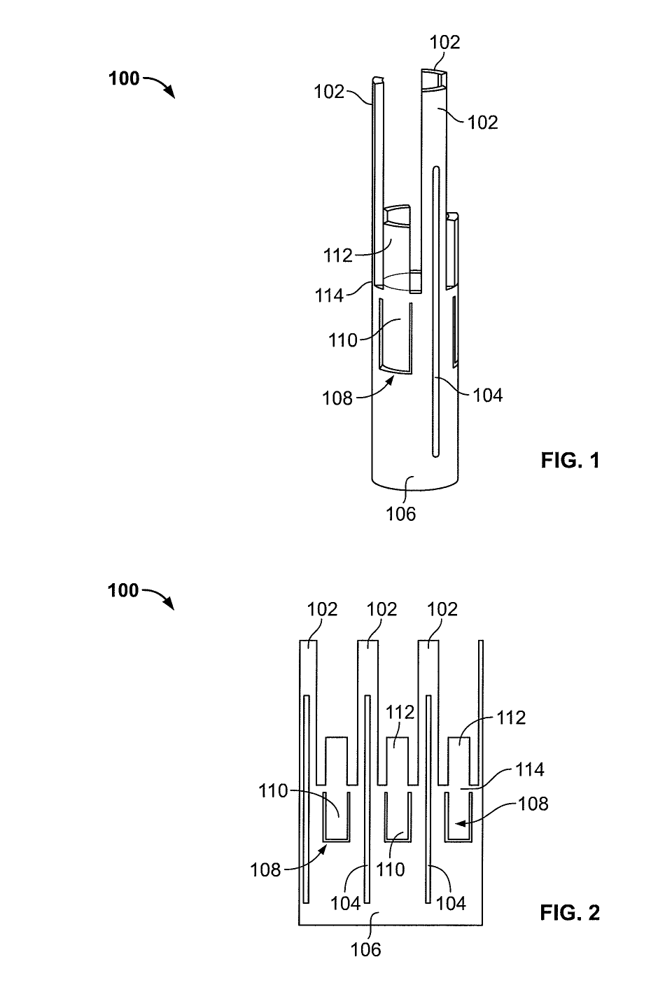

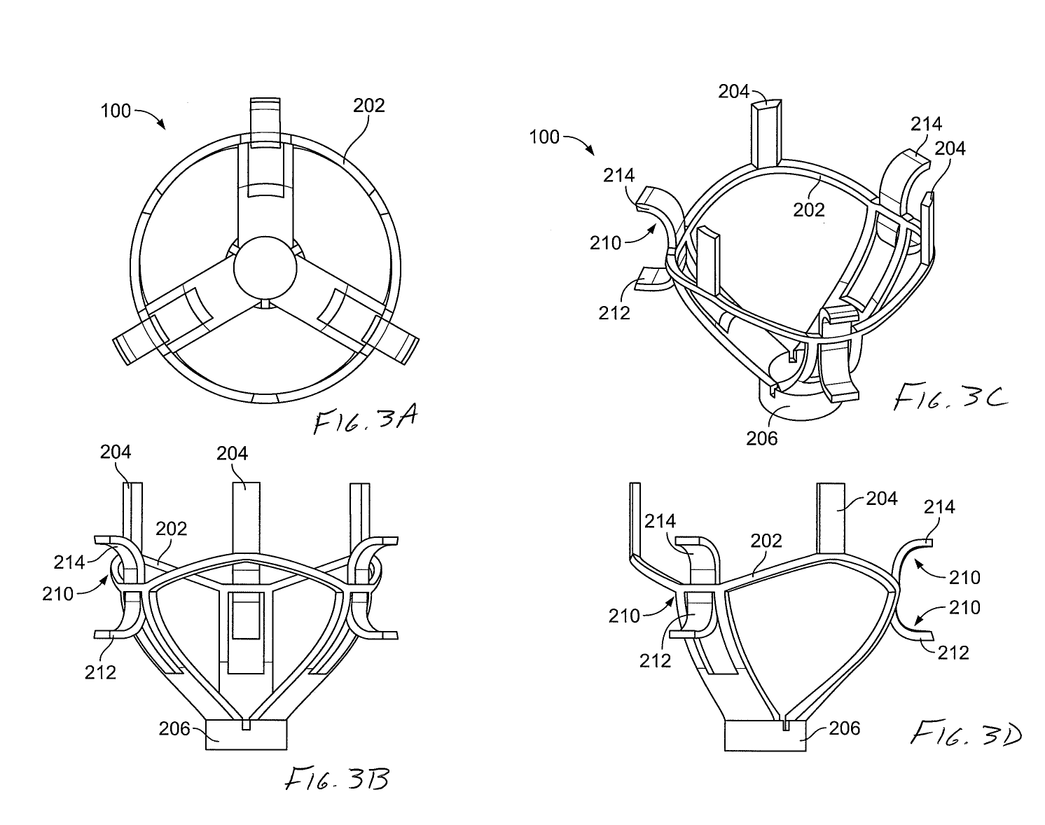

With initial reference to FIGS. 1 and 2, support structure 100 is formed from a tubular member fabricated from a shape memory metal, typically a medical grade of Nitinol. A series of pre-programmed cuts are applied to the tubular member so as to define three (3) spaced, upwardly extending portions 102 with elongated slots 104 formed therein. Slots 104 are bounded at a bottom extent by a band region 106 that extends circumferentially around the tubular member. Between adjacent slots, three upwardly-directed U-shaped cutouts 108 are formed in the tubular member. Each U-shaped cutout 108 defines a substantially rectangular, lower leaf region 110. Cooperative upper leaf regions 112 of comparable rectangular geometry are defined between adjacent upwardly extending portions 102. An annular ring region 114 is defined between upper and lower leaf regions 112, 110 and extends around the tubular member. However, ring region 114 is interrupted by elongated slots 104.

Turning to the views set forth in FIGS. 3A-3D, the support structure 100 formed through the pre-programmed cuts to the tubular member, as shown in FIGS. 1 and 2, is schematically depicted in an expanded/deployed configuration. Such expanded/deployed configuration may be achieved in a variety of ways, e.g., by sequentially introducing a series of rods of increasing diameters into the tubular member and/or otherwise introducing an expanding force within the diameter of tubular member. Of note, the expansive force is limited to the region above band 106.

With further reference to FIGS. 3A-3D, it is initially noted that a support ring 202 extends circumferentially around support structure 100. Support ring 202 is defined through spreading of upwardly extending portions 102, as permitted by the presence of elongated slots 104, and the co-alignment with annular ring region 114. The portion of upwardly extending portions 102 that are not traversed by elongated slots 104 define upstanding tabs 204. Support ring 202 may be upwardly bowed in the regions between annular ring regions 114. The overall geometry of support ring 202 is generally selected to facilitate interaction with valve leaflets (not pictured), and the presence of upstanding tabs 204 may facilitate mounting of valve leaflets with respect thereto and/or dilation of native leaflets as a replacement valve prosthesis that includes support structure 100 is anatomically introduced.

With further reference to FIGS. 3A-3D, the band region 106 defined in the cutting operation reflected in FIGS. 1 and 2 translates to a mounting collar 206 for purposes of support structure 100. Mounting collar 206 advantageously facilitates coupling and de-coupling of support structure 100 with respect to a delivery structure, as described below. Three (3) support struts 208 extend upwardly from mounting collar 206. The U-shaped cutouts 110 formed in the tubular member to define lower leaf region 110, as shown in FIGS. 1 and 2, permit the formation of a lower jaw/arm 212 of retention clip element 210. The upper leaf region 112, in turn, translates to an upper jaw/arm 214 of retention clip element 210. In this way, support structure 100 defines three (3) annularly spaced retention clip elements 210 that are outwardly directed and adapted to engage an anatomical structure, e.g., a heart annulus. Of note, upper jaws/arms 214 may be referred to as “retention clips proximal” and the lower jaws/arms 212 may be referred to as “retention clips distal”.

Retention clip elements 210 are generally processed to define a curved geometry thereto, as shown in FIGS. 3A-3D. The curved geometry can be applied to retention clip elements 210 through conventional metal processing techniques, as will be readily apparent to persons skilled in the art. Once support structure 100 is in the geometric configuration shown in FIGS. 3A-3D, it is generally processed to impart a “memory” effect based on its fabrication from shape memory metal/Nitinol, i.e., through a conventional annealing process. Thus, support structure 100 is generally adapted to automatically revert to the geometric configuration shown in FIGS. 3A-3D, even if maintained in a constricted and/or distorted configuration for a prolonged period.

Of note and with particular reference to FIG. 2, the disclosed support structure 100 may be fabricated from flat stock, e.g., flat stock Nitinol, and then welded to create a round tubular structure (as shown in FIG. 1). The cuts applied to the tubular and/or flat stock is generally applied through computer controlled, laser cut technologies, although alternative techniques may be employed without departing from the spirit or scope of the present disclosure.

In exemplary embodiments of the present disclosure, retention clip elements 210 are symmetrically positioned around the circumference of support structure 100. Symmetric positioning of retention clip elements 210 may prove advantageous for positioning of the support structure (and associated valve prosthesis) relative to an anatomical structure, e.g., a heart annulus. However, non-symmetric positioning of retention clip elements 210 may be employed without departing from the spirit or scope of the present disclosure. The relative positioning of retention clip elements 210 is effectuated through the relative spacing of cuts applied to the tubular member, as depicted in FIGS. 1 and 2, as will be readily apparent to persons skilled in the art.

Support structure 100 is generally transformed into a valve prosthesis by mounting three leaflet membranes (not shown) to support ring 202. The leaflet membranes are generally configured and dimensioned to be positioned substantially between adjacent retention clip elements 210. Each leaflet membrane generally assumes an inwardly bowed orientation when mounted with respect to the support ring 202. More or fewer of the leaflet membranes may be employed without departing from the spirit or scope of the present disclosure, provided the desired blood flow functionality is achieved. The leaflet membranes may be fabricated from xenograft tissue, e.g., the valve leaflets may be fabricated from standard biologic or artificial prosthetic material, such as cryo- or chemically-preserved bovine pericardium or porcine heart valve tissue. Synthetic membrane materials may also be employed in the fabrication of the leaflet membranes, e.g., fiber-reinforced matrix materials. The leaflet membranes may be secured with respect to the support ring 202 through conventional means, e.g., creation of an annulus and/or cuff that surrounds, in whole or in part, the support ring 202 such that each of the plurality of leaflet membranes extends downwardly with respect to the support ring 202.

Additional structures may be mounted with respect to the disclosed support structure to the disclosed support structure 100 in forming an efficacious valve prosthesis. For example, a valve skirt may extend to a full extent of the support ring 202, e.g., to a full extent of the circumference of the support ring 202. The valve skirt 202 may be formed from a single, contiguous structure, or may be defined by a plurality of adjacent and/or overlapping elements that, together, extend along the circumference of support ring 202. The valve skirt may be fabricated from a variety of substantially flexible and/or pliable materials, e.g., xenographic tissue or a synthetic material that is compatible with blood flow, e.g., a non-thrombogenic material.

As noted above, the disclosed support structure 100 offers many advantages. The support ring 202 provides advantageous hoop strength to a valve prosthesis embodying support structure 100. The ability to fabricate support structure 100 from a single, one-piece tubular member offers significant manufacturing and assembly advantages. Moreover, the “memory” effect associated with the shape memory/Nitinol materials used in fabricating support structure facilitates placement/deployment of a valve prosthesis without the need for mechanical and/or other deployment structures/mechanisms.

The disclosed valve prosthesis and associated delivery structures/methods offer numerous advantages relative to existing systems. For example, the retention clip elements associated with the disclosed valve prosthesis valve include upper and lower arcuate regions that may advantageously function to engage the annulus as well as the wall of the ventricular chamber below the annulus, thereby securely aligning and stabilizing the valve prosthesis (e.g., in a re-deployable manner) relative thereto.

It will be appreciated that the disclosed design and implantation methodology may not require extensive surgery, and that the disclosed retention clip elements may function to provide stable and well aligned implantation, central blood flow, and/or a stable platform for the leaflet membranes. Moreover, positioning may be more precise than with a balloon expandable device, such as a stent. Additionally, and also unlike a stent, the positioning may potentially be repeated (e.g., until the desired implantation position and/or orientation is achieved).

In accordance with exemplary embodiments of the present disclosure, the heart valve prosthesis may be placed squarely at the site of a diseased heart valve, as distinct from certain existing heart valve prosthesis implementations characterized by the use of stents configured for placement in the connecting blood vessels adjacent to and/or near the diseased heart valve, and, as such, are designed to be disposed in spaced relation therewith, whether during or after implantation, or during in situ operation. As a result, the ability of the operator or surgeon to reposition and/or re-anchor the heart valve prosthesis in order to more accurately position the heart valve prosthesis in the opening of the diseased heart valve, such as may be provided in accordance with embodiments of the present disclosure, may be of increased significance.

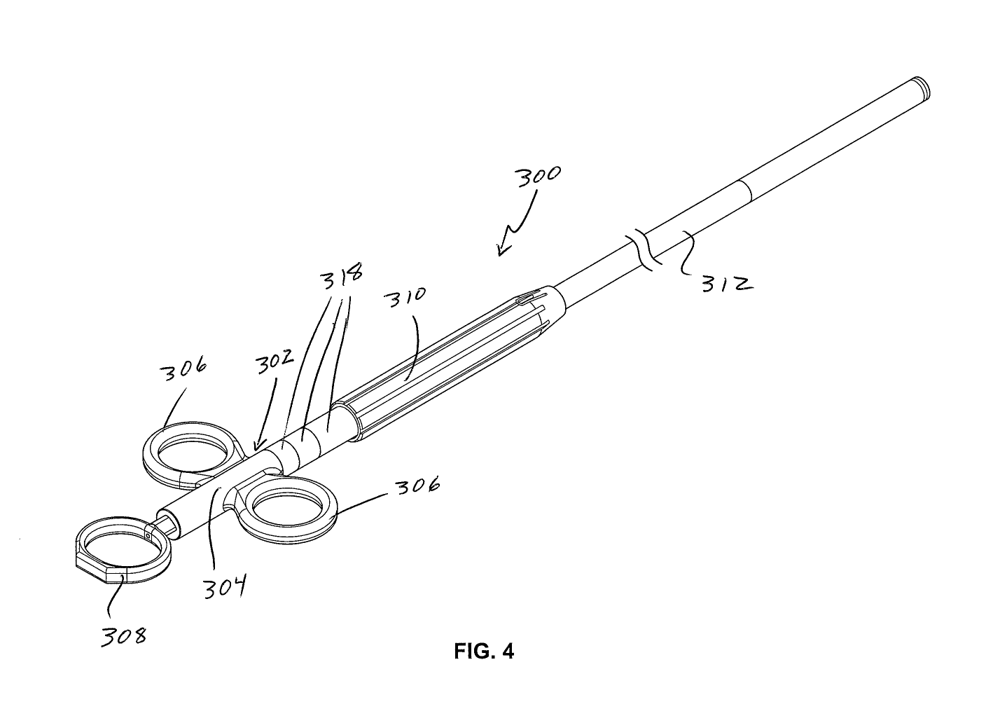

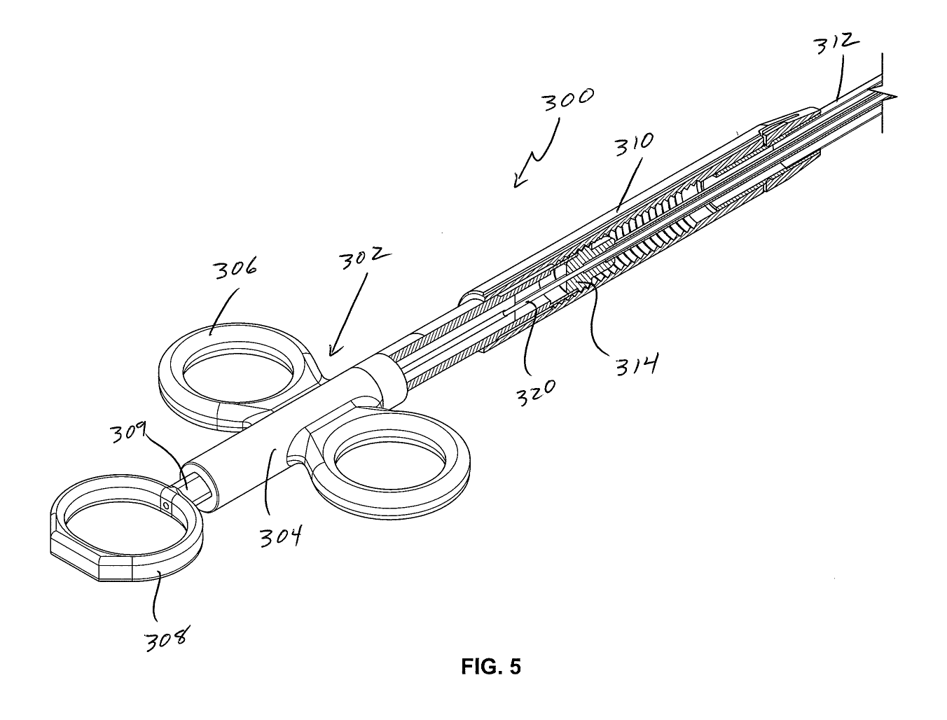

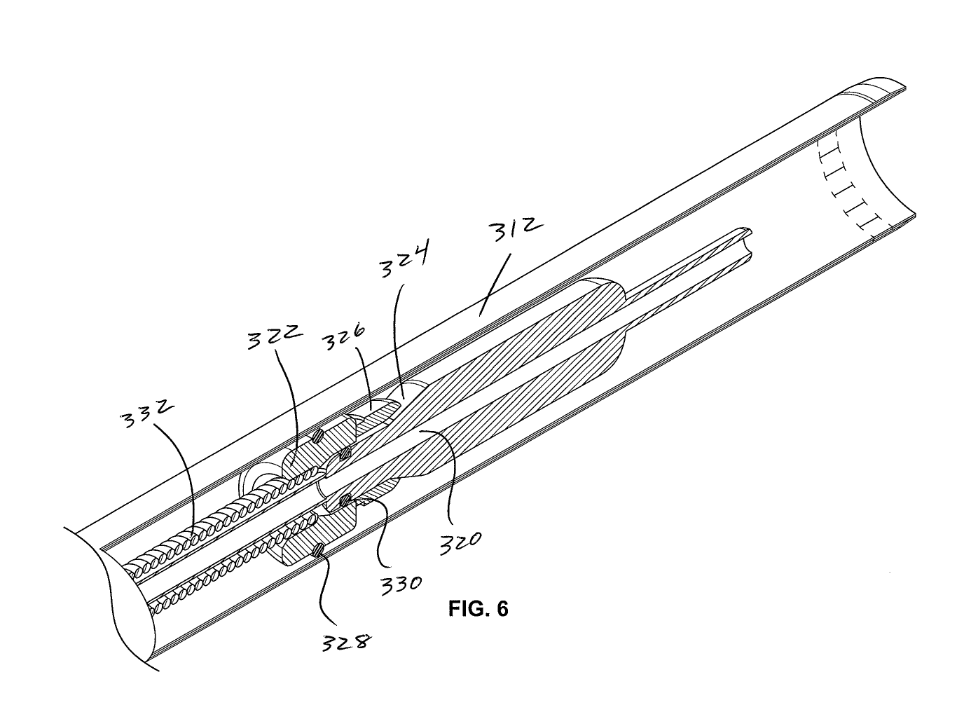

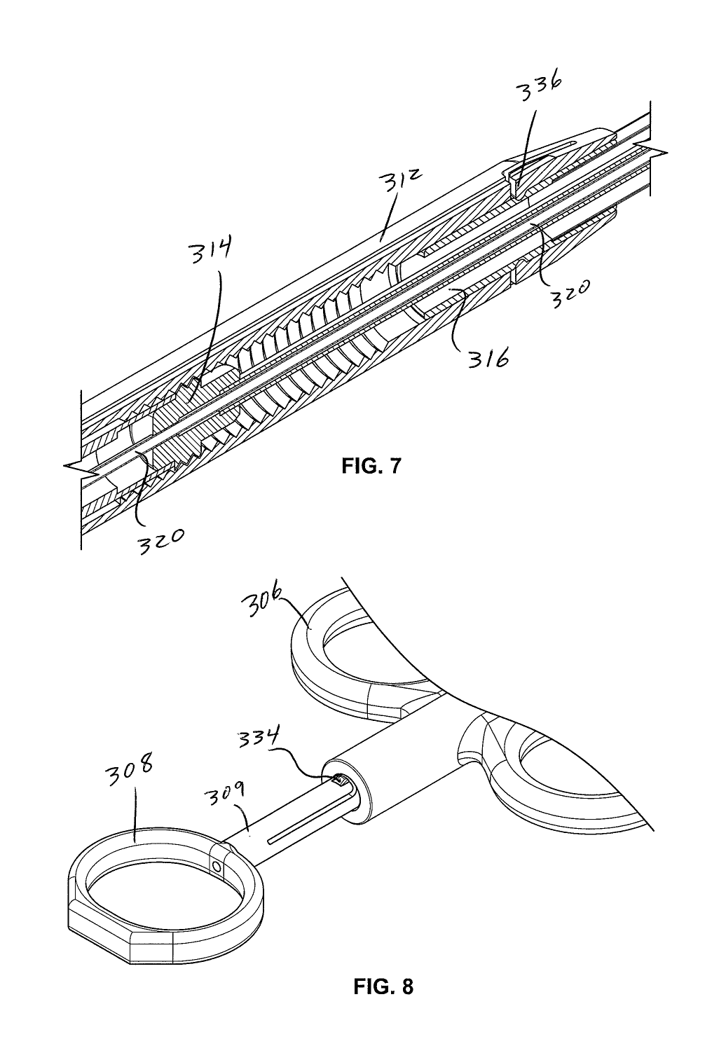

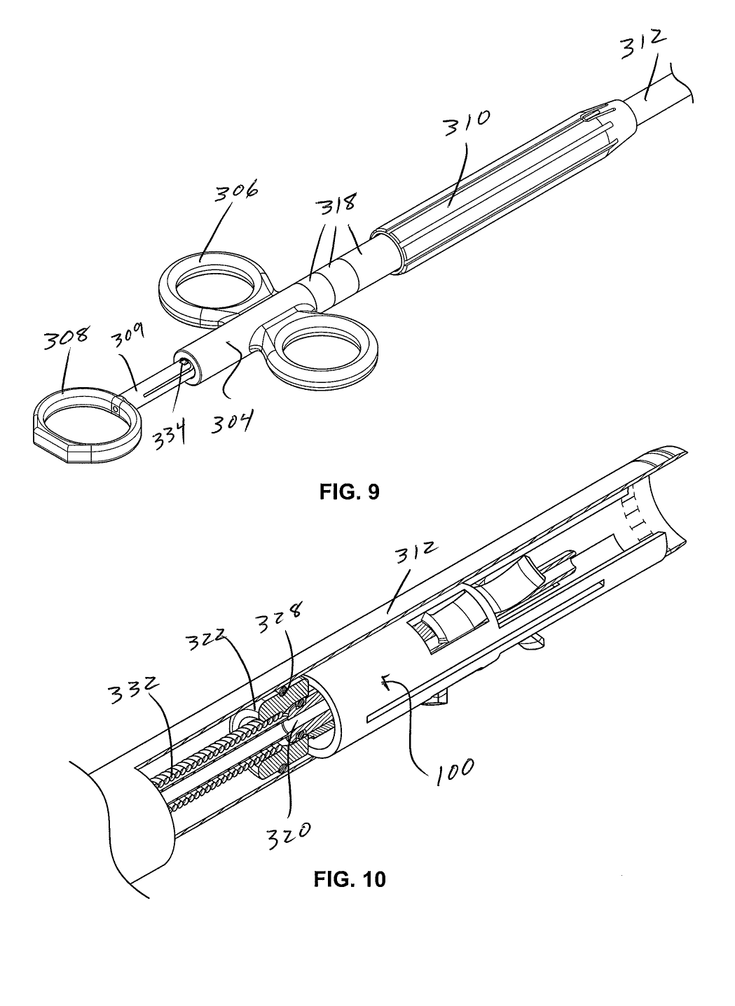

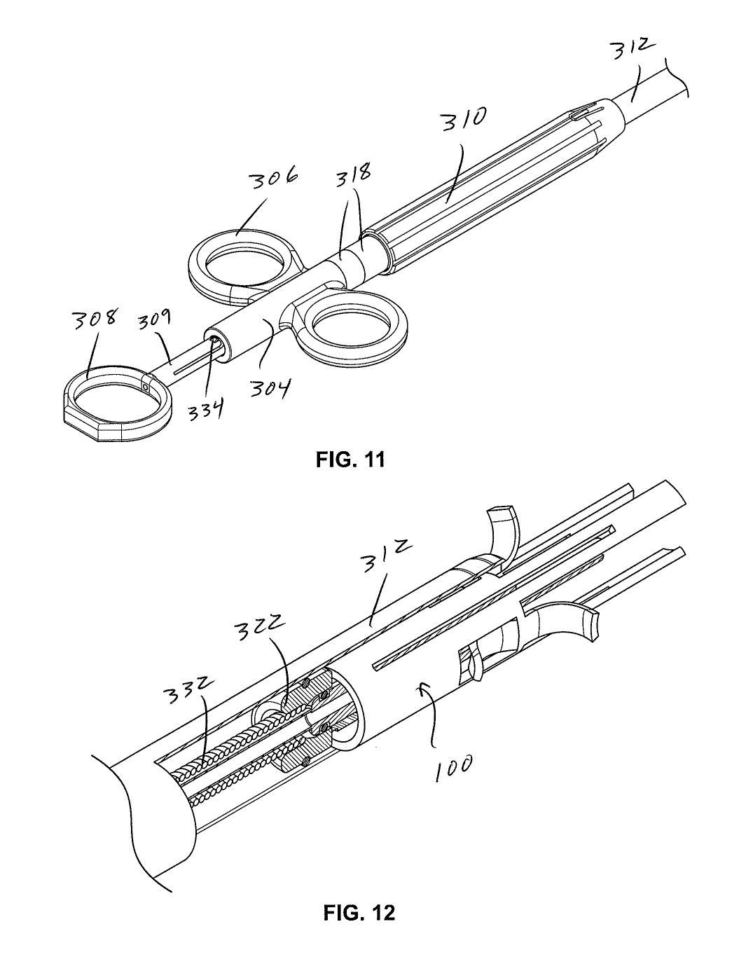

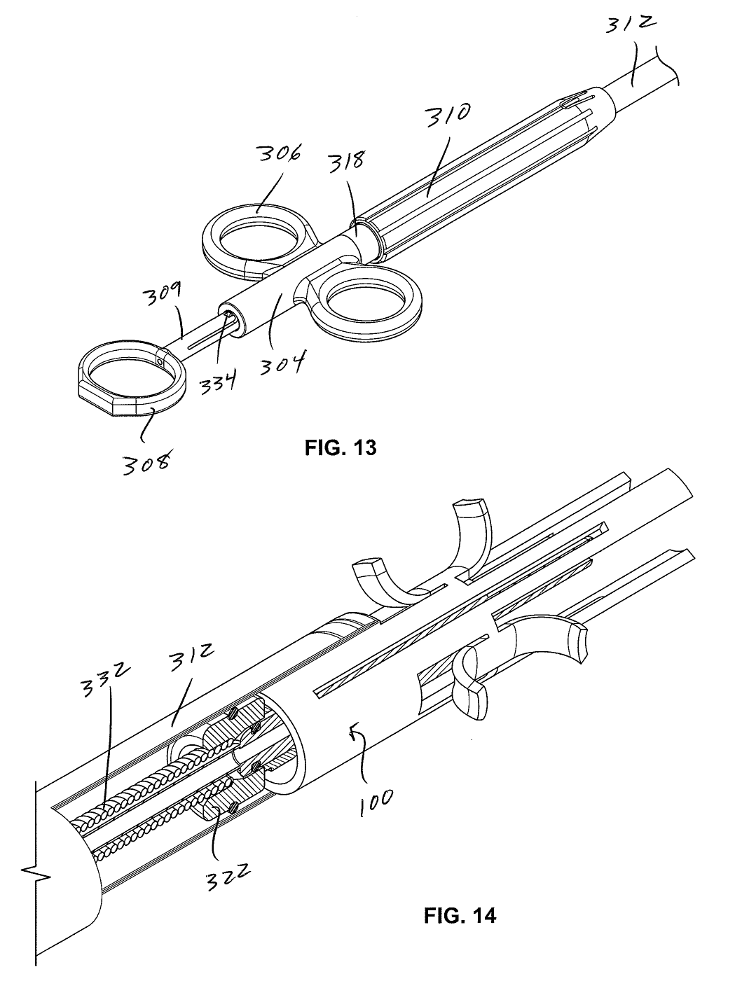

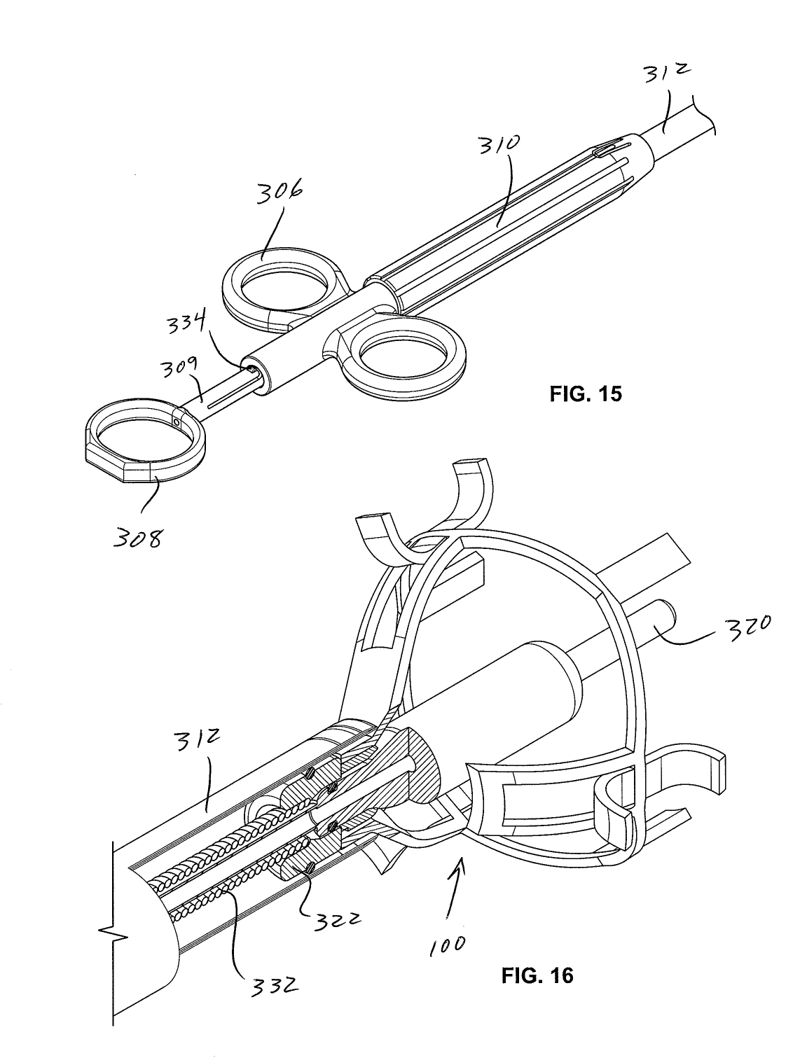

The support structure and associated valve prosthesis of the present disclosure may be implemented by one or more of a plurality of variations, including through use of a valve delivery system of the type depicted in FIGS. 4-16. The exemplary delivery system 300 disclosed herein includes the following structural elements:
1. Body Housing 302 that includes main delivery system handle 304 with finger loops 306.
2. Lock Plunger 308 that cooperates with plunger sleeve 309 and is used to lock and release the valve prosthesis 100 relative to the delivery system 300.
3. Sheath Advance Housing 310 that is used to advance and retract sheath 312.
4. Sheath Advance Ferrule 314 that takes the form of a threaded plug and is used to advance and retract the sheath advance housing 310.
5. Sheath Flange 316 that connects sheath 312 to sheath advance housing 310.
6. Advancement Marking Bands 318 (e.g., color-coded markings/regions) that are used to show staging of valve deployment.
7. Guide Wire Channel Tube 320 that is typically fabricated from Nitinol and is used for accurate clinical placement/positioning of delivery system 300 and associated valve prosthesis 100.
8. Valve Retaining Collet 322 that is used to detachably affix valve tripod/support structure 100 relative to delivery system 300.
9. Retaining Cam 324 defined by guide wire channel tube 320 that is used to spread the distally-facing jaws 326 of valve retaining collet 322 to detachably affix valve tripod/support structure 100 relative to delivery system 300.
10. O-Ring Primary 328 that is used to seal between sheath 312 and valve retaining collet 322.
11. O-Ring Secondary 330 that is used to seal between valve retaining collet 322 and an enlarged portion of guide wire channel 320.
12. Flex Shaft 332 that generally takes the form of a flexible wire coiled shaft and is used to connect valve retaining collet 322 and sheath advance ferrule 314.
13. Proximal detent 334 that extends upwardly from plunger sleeve 309 and engages main delivery system handle 304 to fix the relative position therebetween.
14. Distal detent 336 that extends inwardly from sheath 312.

Having identified key components of exemplary delivery system 300 with reference to

FIGS. 4-16, it is noted that the disclosed delivery system 300 includes two principal subassemblies, an outer sheath assembly and a valve retaining system.

The outer sheath system generally includes the disclosed sheath advance housing, sheath flange, inner sheath (which may be advantageously fabricated from PTFE), flex shaft/coil (which may be fabricated from a suitable stainless steel), outer sheath (which may be fabricated from a thermoplastic polyurethane elastomer, e.g., Pellethane (Dow Chemical) elastomer), and marking bands. The inner sheath, outer sheath and marking bands may be laminated together to form a flexible, kink-resistant protective sheath. The sheath and sheath flange may then be glued or otherwise adhered to each other to form a sheath assembly. The sheath assembly may then be pressed into the sheath advance housing and held in place by the detents at the distal end of the sheath advance housing (see FIG. 7). The noted detents function to retain the sheath assembly with respect to the sheath advance housing and to allow the sheath advance housing to freely spin while maintaining the sheath assembly in a stationary position.

The valve retaining system includes a plunger channel assembly and a retaining system body which include the body housing, lock plunger, sheath advance ferrule, guide wire channel tube, valve retaining collet, retaining cam, O-rings (primary and secondary) and flex shaft. In manufacture, the valve retaining collet is welded to the distal end of the flex shaft. The distal end of the sheath advance ferrule is welded to the proximal end of the flex shaft. The proximal end of the sheath advance ferrule is pressed and glued/adhered to the distal end of the body housing (see FIG. 7) creating a retaining system body. The retaining cam is welded on to the distal end of the guide wire channel tube (Nitinol) and then slipped into the core of the retaining system body. The lock plunger is pressed and glued/adhered to the proximal end of the guide wire channel tube creating the plunger channel assembly and valve retaining system. The sheath system is screwed onto the sheath advance ferrule of the valve retaining system and positioned by aligning the proximal end of the sheath advance housing of the sheath system and the proximal end of the appropriate band on the body housing (shown in FIG. 15), thereby providing the overall valve delivery system.

In terms of valve deployment and with reference to FIGS. 4-16 (which omit the valve leaflets for clarity), the sheath system is initially positioned at the proximal end of an indicating ring of the body housing (see FIG. 15). So positioned, the replacement valve prosthesis (that typically embodies the disclosed support structure 100 fabricated from a single tubular/flat stock member) may be detachably mounted with respect to the valve retaining collet. The mounting collar associated with the valve prosthesis facilitates such mounting engagement.

Thereafter, the plunger channel assembly may be pulled proximally until a detent is exposed and locked in place (illustrated in FIG. 8). This action allows the retaining cam to splay the valve retaining collet radially outward, thereby allowing the tripod/support structure of the valve prosthesis to be retained on the delivery system.

The sheath advancement housing may then be rotated (clockwise in the exemplary embodiment depicted herein) until the distal end of an indicating ring on the body housing is exposed (illustrated in FIG. 9). This action allows the sheath system to advance distally, thereby collapsing the tripod/support structure and associated valve membranes (and any associated structures, e.g., valve skirt). In this way, the replacement valve prosthesis is covered by the delivery system (illustrated in FIG. 10).

At this stage, the delivery system is loaded and ready to be advanced down the catheter sheath. A radiolucent marker band is typically located on the distal end of the sheath system to facilitate fluoroscopic visualization of the sheath system, e.g., the distal end thereof Once the desired location is reached, the disclosed delivery system is ready for valve prosthesis deployment.

To deploy the valve prosthesis, the sheath advancement housing is rotated counter clockwise until the distal end of an indicator ring is exposed (illustrated in FIG. 11). This action exposes the valve leaflets (not shown) and facilitate deployment of the distal retention clip elements (FIG. 1) of the tripod/support structure (illustrated in FIG. 12).

By rotating the sheath advancement housing counterclockwise until the distal end of an indicator ring is exposed (illustrated in FIG. 13), this action deploys the proximal retention clip elements (FIG. 1) of the tripod/support structure (illustrated in FIG. 16).

By rotating the sheath advancement housing counterclockwise to the proximal end of an indicator ring (illustrated in FIG. 15), this action allows the tripod/support structure of the valve prosthesis to be fully deployed (based on its shape memory properties) and locked in place (illustrated in FIG. 16).

Of note, if proper placement of the valve prosthesis is not initially achieved (or the clinician desires to reposition the valve prosthesis for any reason), the valve prosthesis can be repositioned by rotating the sheath advancement housing clockwise, thereby allowing the tripod/support structure to again collapse and to thereby be dislodged from anatomical/annulus engagement, e.g., for repositioning.

Once proper positioning is established, the detent on the lock plunger may be depressed to release the valve prosthesis from the delivery system.

Although implementations of the invention have been described in detail above, those skilled in the art will readily appreciate that many additional modifications are possible without materially departing from the novel teachings and advantages of the invention. Any such modifications are intended to be included within the scope of the invention as defined in the following claims.

1. A one-piece support structure, comprising:
a substantially cylindrical structure that defines (a) a support ring; (b) a mounting collar;
(c) a plurality of support struts extending from the mounting collar to the support ring; and (d) a plurality of retention clip elements, wherein a retention clip element is defined adjacent each of the plurality of support struts;
wherein the supporting ring, mounting collar, plurality of support struts and plurality of retention clip elements are formed by applying cuts to a one-piece structure.

2. The one-piece support structure according to claim 1, wherein the one-piece structure is a tubular member.

3. The one-piece support structure according to claim 1, wherein the one-piece structure is flat stock that is processed to form a tubular member after the cuts are applied.

4. The one-piece support structure according to claim 1, wherein the one-piece structure is fabricated from a shape memory metal.

5. The one-piece support structure according to claim 1, wherein the retention clip elements define upper and lower regions.

6. The one-piece support structure according to claim 1, further comprising a plurality of leaflet members mounted with respect to the support ring.

7. The one-piece support structure according to claim 1, wherein the support structure is embodied in a replacement valve prosthesis.

8. The one-piece support structure according to claim 1, wherein the support structure is embodied in a mitral heart valve prosthesis.

9. A method for fabricating a support structure, comprising:
a. providing a tubular member or flat stock of shape memory metal;
b. applying predefined cuts to the tubular member or flat stock and, if working with flat stock, welding the flat stock into a tubular member; and
c. expanding the tubular member to an expanded configuration to define a support structure having a mounting collar, a plurality of support struts, a support ring and a plurality of retention clip elements.

10. The method according to claim 9, further comprising annealing the expanded tubular member to apply a memory effect thereto.

11. The method according to claim 10, further comprising mounting leaflet membranes to the support ring of the support structure.

12. A delivery system for delivering a support structure to a desired location, comprising:
a. a sheath system that includes a sheath advance housing, a sheath flange, an inner sheath, and an outer sheath, and
b. a valve retaining system that includes a body housing, a lock plunger, a sheath advance ferrule, a guide wire channel tube, a valve retaining collet, a retaining cam, a plurality of O-rings and a flex shaft,
wherein the sheath system and the valve retaining system cooperate to facilitate delivery of a support structure to a desired anatomical location.
